(19) United States
(12) Patent Application Publication
(10) Pub. No.: US 2009/0087410 A1
(43) Pub. Date: Apr. 2, 2009

(54) TREATMENT OF HYPERSENSITIVITY

(75) Inventors: Andrew John Hubert Gearing, Victoria (AU); Barbara Jane Johnson, Queensland (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CBio Limited, Eight Mile Plains (AU)

(21) Appl. No.: 12/090,821

(22) PCT Filed: Oct. 20, 2006
(86) PCT No.: PCT/AU2006/001566

§ 371 Date: Sep. 9, 2008

(30) Foreign Application Priority Data
Oct. 20, 2005 (AU) 2005905815

(51) Int. Cl. A61K 38/17; A61K 38/21; A61K 31/436; A61K 31/519; A61K 31/522; A61K 39/395; A61P 43/00

(52) U.S. Cl. 424/85.6; 514/12; 424/85.4; 424/85.7; 514/291; 514/249; 514/263.31; 424/130.1

(57) ABSTRACT

The present invention relates to a method for inhibiting a hypersensitivity reaction in a subject, wherein said method comprises administering an effective amount of chaperonin (10).

### TECHNICAL FIELD

The present invention relates to the treatment of hypersensitivity by administering a therapeutically effective amount of eukaryotic chaperonin10 (Cpn10) and in particular, the invention relates to the use of eukaryotic Cpn10 for treatment of allergic conditions associated with hypersensitivity, including asthma.

### BACKGROUND

The sensitization of an individual to a particular antigen or combination of antigens, whereupon subsequent exposure causes extreme allergic reactions, can be injurious and even fatal. When an adaptive immune response occurs in an exaggerated or inappropriate form, the individual experiencing the reaction is said to be hypersensitive.

Hypersensitivity reactions are the result of immune responses acting inappropriately and can be provoked by many antigens. One form of hypersensitivity occurs when an IgE response is directed against innocuous environmental antigens, such as pollen or dust-mites. The resulting release of pharmacological mediators by IgE-sensitized mast cells produces an acute inflammatory reaction with symptoms such as asthma or rhinitis.

The present invention reflects the surprising discovery that eukaryotic Cpn10 can inhibit hypersensitivity reactions, including asthma.

### SUMMARY

According to a first aspect of the present invention there is provided a method for inhibiting a hypersensitivity reaction in a subject, wherein said method comprises administering an effective amount of eukaryotic chaperonin 10.

According to a second aspect of the present invention there is provided a method for treating or preventing a hypersensitivity reaction associated disorder in a subject, the method comprising administering to the subject an effective amount of eukaryotic chaperonin 10, wherein the chaperonin 10 modulates signalling from a Toll-like receptor.

According to a third aspect of the present invention there is provided a composition for treating or preventing a hypersensitivity reaction associated disorder in a subject, the composition comprising an effective amount of eukaryotic chaperonin 10, together with at least one concomitant therapy.

According to a fourth aspect of the present invention there is provided a method for treating or preventing a hypersensitivity reaction associated disorder in a subject, the method comprising administering an effective amount of the composition in accordance with the third aspect of the invention.

The hypersensitivity reaction may involve the activation of basophils, eosinophils, mast cells, neutrophils and lymphocytes, and may involve activation of Toll-like receptor (TLR) signalling. The hypersensitivity reaction may reflect high levels of eosinophils and immunoglobulin E.

An example of a hypersensitivity reaction is an inflammatory reaction. More specifically, examples of a hypersensitivity reaction include food allergy, dermatitis, allergic conjunctivitis, rhinitis, eczema, anaphylaxis and respiratory diseases associated with airway inflammation. Examples of these respiratory diseases include asthma, such as allergic asthma, intrinsic asthma and occupational asthma.

TLR activation plays an important role in the aetiology of inflammatory lung diseases ranging from ARDS (acute respiratory distress syndrome) to asthma and COPD (chronic obstructive pulmonary disease). The Toll-like receptors may be selected from the group comprising of: TLR1, TLR2, TLR3, TLR4, TLR5, TLR6, TLR7, TLR8, TLR9, and TLR10.

The eukaryotic chaperonin 10 may be a naturally-derived, recombinantly produced or synthetically produced chaperonin 10. The eukaryotic chaperonin 10 may be of mammalian origin. The chaperonin 10 may be human chaperonin 10.

The chaperonin 10 may comprise the polypeptide sequence as set forth in SEQ ID NO:1, 2, 3, 4, 7, 9, 11, 13, 15 or 17. The chaperonin 10 may be acetylated or non-acetylated. The chaperonin 10 may lack or substantially lack, protein folding activity.

The eukaryotic chaperonin 10 may be administered in the form of a polynucleotide encoding chaperonin 10. The polynucleotide encoding chaperonin 10 may be located in a genetic construct, operably linked to a promoter.

The eukaryotic chaperonin 10 may be encoded by a polynucleotide which may comprise the sequence as set forth in SEQ ID NO:5, 6, 8, 10, 12, 14, 16 or 18.

The methods may further comprise the administration of at least one additional agent. The agent may be an immunomodulator. The immunomodulator may be a type I interferon such as IFNα or IFNβ.

The concomitant therapy may include administration of agents, such as anti-inflammatory compounds, bronchodilatory compounds or immunosuppressant agents, or a combination thereof.

The immunosuppressant agent may be an immunosuppressant drug or a specific antibody directed against B or T lymphocytes, an antibody against a cytokine e.g. IL-3 IL-5, IL-13, GM-CSF, or surface receptors that mediate their activation, including IgE, and FcEpsilonReceptor.

The immunosuppressant drug may be chromoglycalates, theophylline, leukotriene antagonist, antihistamine or a combination thereof.

The composition in accordance with the third aspect may further comprise a corticosteroid.

The above aspects and embodiments contemplate the use of wild-type and modified forms of eukaryotic chaperonin 10 including mammalian chaperonin 10, and for example human chaperonin 10, as well as full-length chaperonin 10 polypeptides and fragments or derivatives thereof retaining immunomodulatory activity.

Definitions

In the context of this specification, the term “comprising” means “including principally, but not necessarily solely”. Furthermore, variations of the word “comprising”, such as “comprise” and “comprises”, have correspondingly varied meanings.

As used herein the terms “treatment”, “treating” and variations thereof, refer to any and all uses which remedy a disease state or symptoms, prevent the establishment of disease, or otherwise prevent, hinder, retard, or reverse the progression of disease or other undesirable symptoms in any way whatsoever.

As used herein the term “effective amount” includes within its meaning a non-toxic but sufficient amount of an agent or compound to provide the desired therapeutic or prophylactic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “polypeptide” means a polymer made up of amino acids linked together by peptide bonds. The terms “polypeptide” and “protein” are used interchangeably herein, although for the purposes of the present invention a “polypeptide” may constitute a portion of a full length protein.

The term “polynucleotide” as used herein refers to a single- or double-stranded polymer of deoxyribonucleotide, ribonucleotide bases or known analogues or natural nucleotides, or mixtures thereof.

As used herein the terms “modulating”, “modulates” and variations thereof refer to increasing or decreasing the level of activity, production, secretion or functioning of a molecule in the presence of a particular modulatory molecule or agent of the invention compared to the level of activity, production, secretion or other functioning thereof in the absence of the modulatory molecule or agent. These terms do not imply quantification of the increase or decrease. The modulation may be of any magnitude sufficient to produce the desired result and may be direct or indirect.

The term “immunomodulator” as used herein refers to a molecular mediator secreted by one or more cell types and which plays a role in the activation, maintenance, maturation, inhibition, suppression or augmentation of an immune response.

### BRIEF DESCRIPTION OF THE DRAWINGS AND SEQUENCE LISTING

The present invention will now be described, by way of non-limiting example only, with reference to the accompanying drawings.

FIG. 1. Percent eosinophils out of total cells counted in the bronchoalveolar lavage (BAL) of sheep (n=5 per group) treated with vehicle, 0.5, 4, or 16 mg Cpn10 delivered i.v. (A) or vehicle, 0.5 or 4 mg Cpn10 delivered directly into the left lobe of the lung, with vehicle delivered into the right lobe (B) followed by challenge with house dust mite (HDM). Lavage samples were taken at 48 hrs post HDM challenge.

FIG. 2. A. HDM-specific IgE levels pre-(d0) and post-(d7) HDM challenge in i.v. group following administration of 4 mg Cpn10. B. Serum IgE levels pre-(d0) and post-(d7) HDM challenge in intra-lung group following administration of 4 mg Cpn10.

FIG. 3. Representative bronchiolar airways from two sheep 48 hours after house dust mite (HDM) challenge. The sheep were administered either an intra-lung infusion of vehicle (1), or a single iv injection of 4 mg Cpn10 before HDM challenge (2). Note the infiltrating inflammatory cells which completely surround the airway in the vehicle control sheep (arrow) which are largely absent in the airway of sheep given IV Cpn10 before HDM challenge (x100 H&E).

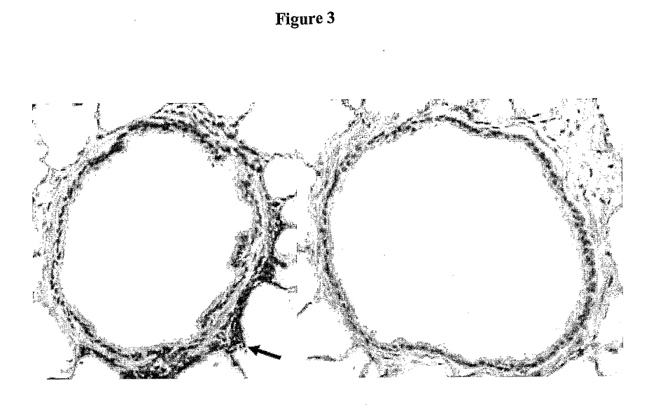

FIG. 4. Representative terminal bronchioles from two sheep 48 hours after house dust mite (HDM) challenge. The sheep were administered either an intra-lung infusion of vehicle (1), or a single iv injection of 4 mg Cpn10 before HDM challenge (2). Note the infiltrating inflammatory cells which surround the airway in the vehicle control sheep (arrow) which is largely absent in the airway of sheep given IV Cpn10 before HDM challenge. (x100 H&E)

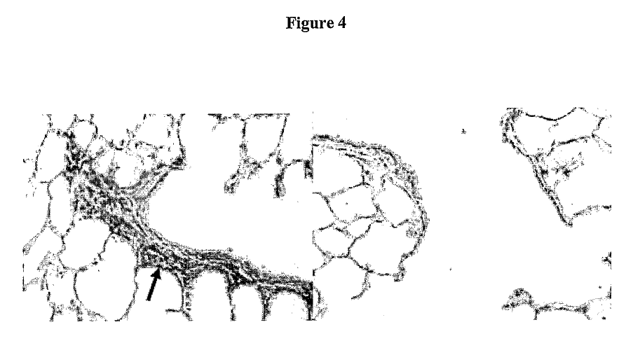

FIG. 5. High power micrograph of a bronchial airway in sheep #6 48 hours after HDM-challenge and administration of an intra-lung of vehicle (control). Note the PAS stained red mucous which completely occludes the bronchial gland lumen (arrow). Also note goblet and epithelial cell hyperplasia and mucous lining the airway lumen. (x400 Alcian blue PAS stained)

FIG. 6. Micrograph of a bronchial airway of sheep #44 48 hours after HDM-challenge and an iv injection of 4 mg Cpn10. Note that the bronchial gland lumens are largely devoid of mucous apart from a small region of blue mucous located in the left side of the gland (arrow). Also note that the goblet and epithelial cell lining the airway lumen are more typical of an unchallenged animal. (x250 Alcian blue PAS stained)

Sequence listing: the amino acid sequence of wild-type human Cpn10 (GenBank Accession No. X75821) is provided in SEQ ID NO:1. The amino acid sequences of two modified forms of Cpn10, with additional amino acid residues at the N-terminal are provided in SEQ ID NOs:2, 3 and 4. The nucleotide sequence encoding the same is provided in SEQ ID NO:5 and 6. Another modified form of wild-type human Cpn10 comprises the deletion of the mobile loop as set forth in SEQ ID NO:7. The nucleotide sequence encoding the same is provided in SEQ ID NO:8. Further to this, other modified forms of wild-type human Cpn10 comprise deletions within the mobile loop as provided in SEQ ID NOs:13, 15 and 17. The nucleotide sequences encoding same is provided in SEQ ID NO:14, 16 and 18. Additional modified forms of wild-type human Cpn10 comprise the deletion of the Beta hairpin roof loop (SEQ ID NO:9) or the deletion of both the mobile loop and the Beta hairpin roof loop (SEQ ID NO: 1). The nucleotide sequences encoding same is provided in SEQ ID NOs:10 and 12.

### DETAILED DESCRIPTION OF PREFERRED EMBODIMENTS

Hypersensitivity reactions are the result of immune responses acting inappropriately and can be provoked by many antigens. One form of hypersensitivity occurs when an IgE response is directed against innocuous environmental antigens, such as pollen or dust-mites. The resulting release of pharmacological mediators by IgE-sensitized mast cells produces an acute inflammatory reaction with symptoms such as asthma or rhinitis.

Airway inflammation is central to the pathogenesis of asthma and involves the recruitment and activation of eosinophils, mast cells, neutrophils and lymphocytes into lung tissue and bronchoalveolar spaces (Busse et al, 2001). House dust mite antigen has been shown to be the most common allergen causing allergic asthma in humans, with sensitized individuals developing specific IgE and IgG humoral responses (Roche et al, 1997). The appropriate animal model of asthma would allow defined and controlled investigations to be conducted with direct relevance to human disease.

The present invention is exemplified by assessing the modulating effects of Cpn10 in a sheep model of asthma. However, it will be appreciated that the concept is applicable to other hypersensitivity reaction type disorders.

Previous sheep models of asthma are based on animals sensitized to nematode (Ascaris) allergens and the results extrapolated to the evaluation of physiological and pharmacological effects of potential anti-asthma drugs. A model of allergic lung inflammation in sheep using house dust mite as an allergen with direct relevance to human allergic disease has recently been established. In this model, sensitized sheep develop allergen-specific IgE responses with recruitment of neutrophils, eosinophils and activated lymphocytes into the lung tissue and BAL with similar kinetics to humans challenged with HDM allergen (Bischof et al, 2003).

The present invention provides a method for inhibiting a hypersensitivity reaction in a subject, wherein said method comprises administering an effective amount of eukaryotic chaperonin 10.

### Cpn10

In accordance with aspects and embodiments of the present invention, a subject in need of treatment is administered with an effective amount of eukaryotic, for example, human Cpn10, also known as heat shock protein 10 kDa (Hsp10). For example, the subject to be treated is a human, and accordingly, the Cpn10 polypeptide is the human Cpn10 polypeptide. Those skilled in the art will appreciate that the precise identity of the Cpn10 used in accordance with the present invention may vary depending on a number of factors, for example the species to be treated, such that the Cpn10 may be selected so as to be derived from the species to be treated.

Typically, the Cpn10 is recombinant Cpn10. Methods described in Morton et al., 2000 (Immunol Cell Biol 78:603-607), Ryan et al., 1995 (J Biol Chem 270:22037-22043) and Johnson et al, 2005 (J Biol Chem 280:4037-4047) are examples of suitable production methods for recombinant Cpn10 protein, although the skilled addressee will appreciate that the present invention is not limited by the method of purification or production used and any other method may be used to produce Cpn10 for use in accordance with the methods and compositions of the present invention.

Eukaryotic Cpn10 polypeptides and peptide fragments for use in accordance with the present invention may be obtained using standard recombinant nucleic acid techniques or may be synthesized, for example using conventional liquid or solid phase synthesis techniques. Cpn10 peptides may be produced by digestion of a polypeptide with one or more proteinases such as endoLys-C, endoArg-C, endoGlu-C and staphylococcus V8-protease. The digested peptide fragments can be purified by, for example, high performance liquid chromatographic (HPLC) techniques.

Embodiments of the invention also contemplate the administration of a polynucleotide encoding eukaryotic Cpn10. In such situations the polynucleotide is typically operably linked to a promoter such that the appropriate polypeptide sequence is produced following administration of the polynucleotide to the subject. The polynucleotide may be administered to subjects in a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences. The nucleic acid construct to be administered may comprise naked DNA or may be in the form of a composition, together with one or more pharmaceutically acceptable carriers.

The eukaryotic Cpn10 polypeptide may have the amino acid sequence as set forth in SEQ ID NO:1, 2, 3, 4, 7, 9, 11, 13, 15 or 17. The nucleotide sequence of the polynucleotide encoding Cpn10 may be as set forth in SEQ ID NO:5, 6, 8, 10, 12, 14, 16 or 18 or display sufficient sequence identity thereto to hybridise to the sequences of SEQ ID NO: 5, 6, 8, 10, 12, 14, 16 or 18. In alternative embodiments, the nucleotide sequence of the polynucleotide may share at least 40%, 50%, 60%, 70%, 80%, 85%, 90%, 96%, 97%, 98% or 99% identity with the sequences set forth in SEQ ID NO:5, 6, 8, 10, 12, 14, 16 or 18.

Within the scope of the terms “polypeptide” and “polynucleotide” as used herein are fragments and variants thereof. By way of example only, peptide fragments of Cpn10 as described in WO 95/15338 and PCT/AU2006/001278, the disclosure of which is incorporated herein by reference, may be used in accordance with aspects and embodiments of the present invention.

The term “fragment” refers to a nucleic acid or polypeptide sequence that encodes a constituent or is a constituent of full-length Cpn10 protein. In terms of the polypeptide the fragment possesses qualitative biological activity in common with the full-length protein. A biologically active fragment of Cpn10 used in accordance with the present invention may typically possess at least about 50% of the immunomodulatory activity of the corresponding full length protein, more typically at least about 60% of such activity, more typically at least about 70% of such activity, more typically at least about 80% of such activity, more typically at least about 90% of such activity, and more typically at least about 95% of such activity.

As further described herein the present inventors have also demonstrated that the addition of a glycine residue to the N terminus of Cpn10 augments immunomodulatory activity. It is contemplated that the presence of an acetyl group or an amino acid which shares structural homology to an acetyl group such as an alanine residue or a glycine residue augments immunomodulatory activity of Cpn10.

The term “variant” as used herein refers to substantially similar molecules. Generally, nucleic acid sequence variants encode polypeptides which possess qualitative biological activity in common. Generally, polypeptide sequence variants also possess qualitative biological activity in common. Further, these polypeptide sequence variants may share at least 40%, 50%, 55%, 60%, 65%, 70%, 75%, 80%, 82%, 85%, 90%, 95%, 96%, 97%, 98% or 99% sequence identity and 70%, 75%, 80%, 82%, 85%, 90%, 95%, 96%, 97%, 98% or 99% sequence similarity.

Further, a variant polypeptide may include analogues, wherein the term “analogue” means a polypeptide which is a derivative of Cpn10, which derivative comprises addition, deletion, substitution of one or more amino acids, such that the polypeptide retains substantially the same function as native Cpn10. It is well known in the art that some amino acids may be changed within a polypeptide without altering the activity of the polypeptide (conservative substitutions). The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution. Amino acid additions may result from the fusion of a Cpn10 polypeptide or fragment thereof with a second polypeptide or peptide, such as a polyhistidine tag, maltose binding protein fusion, glutathione S transferase fusion, green fluorescent protein fusion, or the addition of an epitope tag such as FLAG or c-myc. For example, the wild-type human Cpn10 polypeptide may comprise an additional GSM tripeptide moiety at the N-terminus (SEQ ID NO:2; see for example WO 95/15338, the disclosure of which is incorporated herein by reference) or an additional alanine (A) reside at the N-terminus (SEQ ID NO:3; WO 2004/041300, the disclosure of which is incorporated herein by reference), or an additional glycine (G) at the N-terminus, SEQ ID NO:4 (see for example PCT/AU2006/001278). The wild-type Cpn10 polypeptide may or may not include the initiating methionine at the N terminus. The wild-type Cpn10 polypeptide may be modified at the N-terminus or C-terminus by the addition, deletion, or substitution of one or more amino acid residues.

In another example, the wild-type human Cpn10 polypeptide may lack the mobile loop (SEQ ID No:7), Beta-hairpin roof loop (SEQ ID NO:9) or both (SEQ ID NO:11) which can be observed for example in PCT/AU2006/001278, the disclosure of which is incorporated herein by reference. In addition, the wild-type human Cpn10 polypeptide may contain tripeptide deletions in the mobile loop as set forth in SEQ ID NO: 13, 15 or 17 (see for example PCT/AU2006/001278, the disclosure of which is incorporated herein by reference. The present invention also contemplates the use of polynucleotides encoding such modified forms of Cpn10.

Cpn10 variants can be generated by mutagenesis of a Cpn10 protein or mutagenesis of an encoding nucleic acid, such as by random mutagenesis or site-directed mutagenesis using methods well known to those skilled in the art. Such methods may be found, for example in Current Protocols In Molecular Biology (Chapter 9), Ausubel et al., 1994, John Wiley & Sons, Inc., New York, the disclosure of which is incorporated herein by reference. Variants and analogues also encompass polypeptides complexed with other chemical moieties, fusion proteins or otherwise post-transitionally modified. Examples of suitable modifications are described in International Patent Application No. PCT/AU2005/000041, the disclosure of which is incorporated herein by reference.

Further, the Cpn10 polypeptide or fragment thereof may possess other post-translational modifications, including side-chain modifications such as for example acetylation, amidination, carbamoylation, reductive alkylation and other modifications as are known to those skilled in the art.

A further example of a chaperonin 10 variant is one which lacks or substantially lack, protein folding activity, and examples of such modifications are described in International (PCT) Patent Application Number PCT/AU2006/001278, the disclosure of which is incorporated herein by reference.

### Production of Cpn10

In accordance with the present invention Cpn10 polypeptides may be produced using standard techniques of recombinant DNA and molecular biology that are well lo known to those skilled in the art. Guidance may be obtained, for example, from standard texts such as Sambrook et al., Molecular Cloning: A Laboratory Manual, Cold Spring Harbor, N.Y., 1989 and Ausubel et al., Current Protocols in Molecular Biology, Greene Publ. Assoc. and Wiley-Intersciences, 1992. Methods described in Morton et al., 2000 (Immunol Cell Biol 78:603-607), Ryan et al., 1995 (J Biol Chem 270:22037-22043) and Johnson et al., 2005 (J Biol Chem 280:4037-4047) are examples of suitable purification methods for Cpn10 polypeptides, although the skilled addressee will appreciate that the present invention is not limited by the method of purification or production used and any other method may be used to produce Cpn10 for use in accordance with the methods and compositions of the present invention. Cpn10 peptides may be produced by digestion of a polypeptide with one or more proteinases such as endoLys-C, endoArg-C, endoGlu-C and staphylococcus V8-protease. The digested peptide fragments can be purified by, for example, high performance liquid chromatographic (HPLC) techniques.

The purification of Cpn10 polypeptides of the invention may be scaled-up for large-scale production purposes. For example, as described herein the present inventors have developed a bioprocess for the production of large (gram) quantities of highly pure, clinical grade Cpn10 polypeptides by batch fermentation in E. coli.

Cpn10 polypeptides of the present invention, as well as fragments and variants thereof, may also be synthesised by standard methods of liquid or solid phase chemistry well known to those of ordinary skill in the art. For example such molecules may be synthesised following the solid phase chemistry procedures of Steward and Young (Steward, J. M. & Young, J. D., Solid Phase Peptide Synthesis. (2nd Edn.) Pierce Chemical Co., Ill., USA (1984).

In general, such a synthesis method comprises the sequential addition of one or more amino acids or suitably protected amino acids to a growing peptide chain. Typically, either the amino or carboxyl group of the first amino acid is protected by a suitable protecting group. The protected amino acid is then either attached to an inert solid support or utilised in solution by adding the next amino acid in the sequence having the complimentary (amino or carboxyl) group suitably protected and under conditions suitable for forming the amide linkage. The protecting group is then removed from this newly added amino acid residue and the next (protected) amino acid is added, and so forth. After all the desired amino acids have been linked, any remaining protecting groups, and if necessary any solid support, is removed sequentially or concurrently to produce the final polypeptide.

Amino acid changes in Cpn10 polypeptides may be effected by techniques well known to those persons skilled in the relevant art. For example, amino acid changes may be effected by nucleotide replacement techniques which include the addition, deletion or substitution of nucleotides (conservative and/or non-conservative), under the proviso that the proper reading frame is maintained. Exemplary techniques include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction.

The generation of immunomodulatory activity by the Cpn10 polypeptides of the invention may involve the formation of heptamers of the Cpn10 polypeptides. Testing of immunomodulatory activity for the purposes of the present invention may be via any one of a number of techniques known to those of skill in the art. As exemplified herein immunomodulatory activity of Cpn10 polypeptides may be determined by measuring the ability of the polypeptide to modulate signalling from the Toll-like receptor TLR4, for example using a luciferase bioassay, and typically in the presence of a TLR4 agonist such as lipopolysaccharide. Alternatively or in addition, immunomodulatory activity may be determined using other assays in vitro, ex vivo or in vivo, for example via measurement of NF-κB production or the production of cytokines in cells such as peripheral blood mononuclear cells.

### Compositions and Routes of Administration

In general, suitable compositions for use in accordance with the methods of the present invention may be prepared according to methods and procedures that are known to those of ordinary skill in the art and accordingly may include a pharmaceutically acceptable carrier, diluent and/or adjuvant.

In accordance with the present invention, a Cpn10 composition may be prepared comprising Cpn10 alone or as a pharmaceutical composition comprising a pharmaceutically acceptable carrier, adjuvant and/or diluent. Alternatively, the Cpn10 composition may further comprise an immunosuppressant agent.

The immunosuppressant agent may be an immunosuppressant drug or a specific antibody directed against B or T lymphocytes, or surface receptors that mediate their activation. For example, the immunosuppressant drug may be cyclosporine, tacrolimus, sirolimus, mycophenolate mofetil, methotrexate, chromoglycalates, theophylline, leukotriene antagonist or antihistamine, or a combination thereof.

In addition, the pharmaceutical composition for use in accordance with the invention may still further comprise a steroid, such as a corticosteroid.

Compositions may be administered by standard routes. In general, the compositions may be administered by the parenteral (e.g., intravenous, intraspinal, subcutaneous or intramuscular), oral or topical route. Administration may be systemic, regional or local. The particular route of administration to be used in any given circumstance will depend on a number of factors, including the nature of the condition to be treated, the severity and extent of the condition, the required dosage of the particular compound to be delivered and the potential side-effects of the compound.

In general, suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form (such as liquid or powder) suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations of the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

### Dosages

For the purposes of the present invention molecules and agents may be administered to subjects as compositions either therapeutically or preventively. In a therapeutic application, compositions are administered to a patient already suffering from a disease, in an amount sufficient to cure or at least partially arrest the disease and its complications. The composition should provide a quantity of the molecule or agent sufficient to effectively treat the patient.

The therapeutically effective dose level for any particular patient will depend upon a variety of factors including: the disorder being treated and the severity of the disorder; activity of the molecule or agent employed; the composition employed; the age, body weight, general health, sex and diet of the patient; the time of administration; the route of administration; the rate of sequestration of the molecule or agent; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

### Cpn10 Agonists and Antagonists

The present invention also contemplates the use of agonists and antagonists of Cpn10 and methods of screening and producing such agonists and antagonists.

Cpn10 agonists and antagonists may be specifically designed or screened according to their effect upon TLR signalling and immunomodulator secretion.

Antibodies may act as agonists or antagonists of Cpn10, or fragments or analogues thereof. Preferably suitable antibodies are prepared from discrete regions or fragments of the Cpn10 polypeptide, in particular those involved in conferring protease activity and/or partner or substrate binding. An antigenic Cpn10 polypeptide contains at least about 5, and preferably at least about 10, amino acids.

Methods for the generation of suitable antibodies will be readily appreciated by those skilled in the art. For example, an anti-Cpn10 monoclonal antibody, typically containing Fab portions, may be prepared using the hybridoma technology described in Antibodies—A Laboratory Manual, Harlow and Lane, eds., Cold Spring Harbor Laboratory, N.Y. (1988).

In essence, in the preparation of monoclonal antibodies directed toward Cpn10, or fragment or analogue thereof, any technique that provides for the production of antibody molecules by continuous cell lines in culture may be used. These include the hybridoma technique originally developed by Kohler et al., 1975, Nature, 256:495-497, as well as the trioma technique, the human B-cell hybridoma technique (Kozbor et al., 1983, Immunology Today, 4:72), and the EBV-hybridoma technique to produce human monoclonal antibodies (Cole et al, in Monoclonal Antibodies and Cancer Therapy, pp. 77-96, Alan R. Liss, Inc., (1985)). Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980).

In summary, a means of producing a hybridoma from which the monoclonal antibody is produced, a myeloma or other self-perpetuating cell line is fused with lymphocytes obtained from the spleen of a mammal hyperimmunised with a recognition factor-binding portion thereof, or recognition factor, or an origin-specific DNA-binding portion thereof. Hybridomas producing a monoclonal antibody useful in practicing this invention are identified by their ability to immunoreact with the present recognition factor and their ability to inhibit specified transcriptional activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Similarly, there are various procedures known in the art which may be used for the production of polyclonal antibodies. For the production of anti-Cpn10 polyclonal antibody, various host animals can be immunized by injection with Cpn10, or a fragment or analogue thereof, including but not limited to rabbits, chickens, mice, rats, sheep, goats, etc. Further, the Cpn10 polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the antibody may be detected by virtue of its binding with a secondary antibody or reagent which is appropriately labelled. A variety of methods are known in the art for detecting binding in an immunoassay and are within the scope of the present invention.

The antibody (or fragment thereof) raised against Cpn10 or a fragment or analogue thereof has binding affinity for Cpn10. Preferably, the antibody (or fragment thereof) has binding affinity or avidity greater than about 10⁵ M⁻¹, more preferably greater than about 10⁶ M⁻¹, more preferably still greater than about 10⁷ M⁻¹ and most preferably greater than about 10⁸M⁻¹.

In terms of obtaining a suitable amount of an antibody according to the present invention, one may manufacture the antibody(s) using batch fermentation with serum free medium. After fermentation the antibody may be purified via a multistep procedure incorporating chromatography and viral inactivation/removal steps. For instance, the antibody may be first separated by Protein A affinity chromatography and then treated with solvent/detergent to inactivate any lipid enveloped viruses. Further purification, typically by anion and cation exchange chromatography may be used to remove residual proteins, solvents/detergents and nucleic acids. The purified antibody may be further purified and formulated into 0.9% saline using gel filtration columns. The formulated bulk preparation may then be sterilised and viral filtered and dispensed.

Agonists and antagonists other than antibodies are also contemplated. A candidate agonist or antagonist may be identified by an ability to form a molecular complex with one or more of the TLRs, and/or their adaptor molecules as an agonist. Further, a candidate antagonist may be identified by an ability to prevent or disrupt formation of a molecular complex comprising Cpn10, and one or more of the TLRs and/or their adaptor molecules to act as an antagonist.

Techniques and procedures for identifying and producing agonists and antagonists are well known to those skilled in the art, including screening of libraries of molecules such as synthetic chemical libraries such as combinatorial libraries, computer assisted screening of structural databases, computer-assisted modelling and/or design, or more traditional biophysical techniques which detect molecular binding interactions.

The present invention will now be described with reference to specific examples, which should not be construed as in any way limiting the scope of the invention.

### EXAMPLES

### Example 1

### Efficacy Testing of Cpn10 in a Sheep House Dust Mite Asthma Model

Airway inflammation is central to the pathogenesis of asthma and involves the recruitment and activation of eosinophils, mast cells, neutrophils and lymphocytes into lung tissue and bronchoalveolar spaces (Busse et al, 2001). House dust mite has been shown to be the most common allergen causing allergic asthma in humans, with sensitized individuals developing specific IgE and IgG humoral responses (Roche et al, 1997). The appropriate animal model of asthma would allow defined and controlled investigations to be conducted with direct relevance to human disease.

Previous sheep models of asthma are based on animals sensitized to nematode (Ascaris) allergens and the results extrapolated to the evaluation of physiological and pharmacological effects of potential anti-asthma drugs. A model of allergic lung inflammation in sheep using house dust mite as an allergen with direct relevance to human allergic disease has recently been established. In this model, sensitized sheep develop allergen-specific IgE responses with recruitment of neutrophils, eosinophils and activated lymphocytes into the lung tissue and BAL with similar kinetics to humans challenged with HDM allergen (Bischof et al, 2003).

The experiments described herein considered whether Cpn10 administered via intravenous injection or directly instilled into the lung can affect the clinical and immunologic manifestations of allergic response to house dust mite challenge in sensitized sheep.

### Materials & Methods

Sheep (n=10) were immunized with solubilized house dust mite extract and selected for high allergen-specific IgE responses and bronchoalveolar lavage (BAL) eosinophilia. Animals were then treated with either Cpn10 (0.5, 4, or 16 mg) or vehicle delivered via intravenous injection (n=5), or direct delivery into one side of the lung and vehicle into the other side of the lung prior to challenge with HDM using fibre-optic bronchoscopy. Bronchoalveolar lavage fluid and blood samples were collected at 6 and 48 hrs and 7 days post HDM challenge for enumeration of differential cell counts, BAL cytokine quantitation, and serum IgE quantitation. BAL cells were also stored in RNA isolation medium (Trizol) and frozen for subsequent RT-PCR analysis.

### Results

In general, the results presented herein show that Cpn10 has a dramatic and dose-responsive effect on allergic inflammation in a sheep model of asthma.

### Intravenous Delivery of Cpn10

The intravenous administration of Cpn10 resulted in a reduction of BAL neutrophils at the 6 h time-point in a dose-dependent manner. The number of neutrophils present in BAL 6 h after the 2nd HDM challenge alone, without Cpn10 treatment, was an average of 34% between all 6 sheep in the intravenous group. The percentage of neutrophils, expressed as a group average, declined with increasing dose of Cpn10. At the 6 hour time-point, the 0.5 mg, 4 mg and 16 mg doses of Cpn10 resulted in 15%, 12% and 8% neutrophils, in BAL fluid, respectively.

The intravenous administration of Cpn10 resulted in a reduction of BAL eosinophils at the 48 h time-point in a dose-dependent manner. The number of eosinophils present 48 h after the 2nd HDM challenge alone, without Cpn10 treatment, was an average of 31% between all 6 sheep in the intravenous group. The percentage of eosinophils, expressed as a group average, generally declined with increasing dose of Cpn10. At the 48 h time-point, the 0.5 mg, 4 mg and 16 mg doses of Cpn10 resulted in 15%, 16% and 11% eosinophils, in BAL fluid, respectively.

### Intralung Delivery of Cpn10

The percentages of BAL neutrophils at the 6 and 48 h time-points differed between right and left lungs of individual sheep throughout most treatments. However, there was no consistent trend between the left (Cpn10 treated) and right (vehicle treated) lungs. The percentages of BAL eosinophils at the 6 and 48 h time-points also differed between right and left lungs of individual sheep throughout most treatments. Generally there was no consistent trend between the left (Cpn10 treated) and right (vehicle treated) lungs with the exception of the 4 mg Cpn10 dose at 48 h post-challenge. The percentage of BAL eosinophils in each sheep was lower in the left lung 48 h after HDM challenge and administration of 4 mg (total dose) of Cpn10 to left lung compared to the right control lung. Data averaged from all sheep in the i.l. group, shows that local infusion of 4 mg Cpn10 to the left lung reduced the level of eosinophilia in that lung to 50% of the right lung (control, vehicle treated).

### Baseline IgE Responses

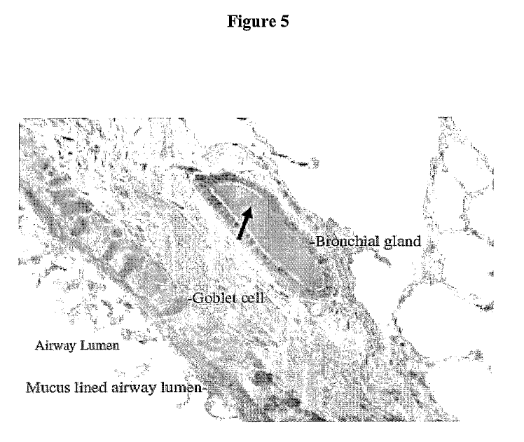

Serum IgE levels were assessed prior to (d0) and seven days after (d7) each of the HDM challenges, as shown in FIG. 5. In the absence of Cpn10 (1st and 2nd HDM challenges), serum IgE levels were clearly elevated following airway challenge with HDM. Sheep #23 was the only sheep that did not show a high IgE response following the initial HDM challenges. Compared to baseline IgE levels before the 1st HDM challenge, slightly elevated d0 IgE levels were noted prior to the 2nd and 3rd HDM challenges; in each case this may reflect a slow return to baseline following HDM challenge 2 weeks earlier.

### IgE Responses to HDM Challenge in the Presence of Cpn10

Cpn10, regardless of dose or mode of delivery, had a marked effect on the HDM-specific serum IgE responses as assessed at 7 days post-HDM-challenge. The administration of Cpn10 resulted in blocking of serum IgE responses, with maintenance at near baseline levels particularly evident with the 4th and 5th HDM challenges.

### Bronchoalveolar Lavage Eosinophils

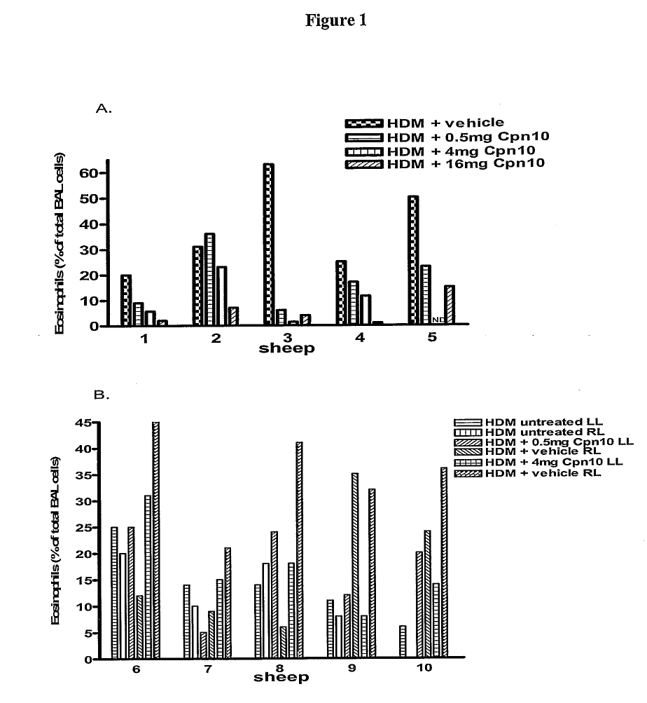

BAL was sampled at baseline (0 h), 6 h and 48 h post-airway HDM challenge and drug treatment and numbers of eosinophils counted. The results to date are graphed in FIG. 1 as percent eosinophils of the total cell population in the lavage fluid sampled at 48 hrs post-HDM challenge.

Intravenous injection of Cpn10 administered prior to HDM challenge results in up to 15-fold reduction in BAL eosinophilia at the peak of eosinophil recruitment at 48 hrs post-challenge.

When Cpn10 is administered unilaterally into the left lung, with vehicle only given to the right (control) lung using a fibre-optic bronchoscope, a 4-fold reduction in the percentage of eosinophils in the BAL from the Cpn10-treated lung vs. the control lung is shown.

### Serum Immunoglobulin Responses to HDM Challenge

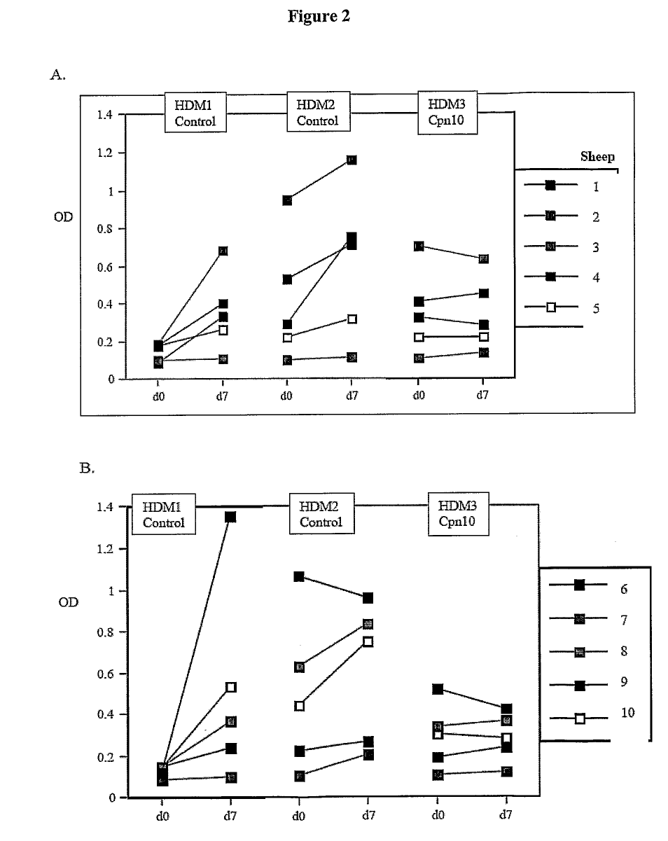

Serum was collected at day 0 and day 7 following administration of either vehicle or Cpn10 and HDM challenge. Sera were then tested and HDM-specific IgE levels assessed using ELISA as detailed in Bischof et al, 2003, with the results presented herein as FIG. 2. The data indicate serum levels of HDM-specific IgE following each of the HDM challenge with vehicle control administration, and following 4 mg Cpn10 and HDM challenge.

### Airway Epithelium, Goblet Cell and Bronchial Gland Changes

### Goblet Cells

Goblet cell hyperplasia is a hallmark feature of asthma pathology which is induced by allergic inflammation of the airways. After allergen provocation, the number of goblet cells per unit length of airway epithelium is known to increase. Concomitant with an increase in number, the goblet cells also increase in size and display large multi-coloured cytoplasms when stained with PAS/Alcian blue histological stains. Analysis of airway epithelium, goblet cells and bronchial glands was performed on PAS/Alcian blue stained histological slides. This analysis was performed on cartilaginous and glandular bronchial airways which were between 1800 and 3300 μm in diameter.

Intra-lung administration of Cpn10 to HDM-challenged sheep: The average number of goblet cells per mm of airway basement membrane was 33 in both left and lungs of intralung administered Cpn10 sheep (n=4 sheep). The goblet cells were large and full of blue and red staining granules (FIG. 5).
Intravenous administration of Cpn10 to HDM-challenged sheep: Compared to the intralung delivery groups, there were fewer goblet cells in bronchial epithelium of sheep treated with 4 mg of Cpn10 via the i.v. route. The average number of goblet cells per mm of airway basement membrane was 16 in sheep administered i.v. Cpn10 (n=6 sheep). This is less than half the number of goblet cells of the vehicle treated intra-lung group (33 goblet cells per mm of airway basement membrane). The goblet cells in the i.v. Cpn10 group were generally less mature in that they were smaller, more cuboidal, and had less cytoplasmic staining.

### Epithelium

The epithelium lining the airways is known to be important tissue component in asthma conditions as it forms a first-line barrier against airborne foreign materials.

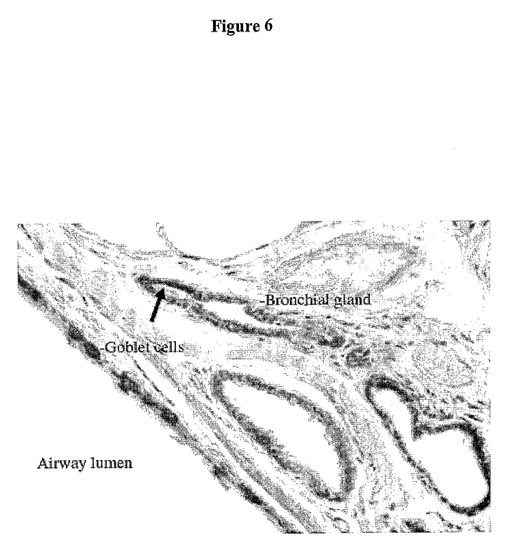

Intra-lung administration of Cpn10 to HDM-challenged sheep: In both the vehicle and Cpn10 intra-lung groups, the airway epithelium was more columnar and in some airways hyperplastic (FIG. 5).
Intravenous administration of Cpn10 to HDM-challenged sheep: In contrast to the intralung group the airway epithelium was more cuboidal (FIG. 6).

### Bronchial Glands

The glands associated with the bronchial airways can be stimulated to secrete mucous into gland lumens after allergen provocation. When stained with PAS/Alcian Blue histological dyes the mucous can stain red (indicating neutral mucins) or blue (indicating acid mucins).

Intra-lung administration of Cpn10 to HDM-challenged sheep: Analysis of PAS/Alcian blue stained histological slides revealed that the bronchial glands of both groups of intralung treated sheep were secreting mucus 48 h after HDM challenge and Cpn10/vehicle treatment (FIG. 5). The mucus in two sheep was PAS stained red (FIG. 5) while in the other two sheep the mucous was predominantly blue. A systematic analysis of all bronchial glands available to be scored in each sheep indicated that on average the bronchial gland lumen was 23% occluded with mucous in vehicle treated lungs and 27% occluded in Cpn10 treated lungs (n=4 sheep).
Intravenous administration of Cpn10 to HDM-challenged sheep: There was markedly less mucous present in the glandular lumens of i.v. treated sheep compared to that observed in the intra-lung groups (FIG. 6). Analysis of PAS/Alcian blue stained histological slides revealed that the bronchial glands of i.v. treated sheep had small amounts of luminal mucus 48 h after HDM challenge and i.v. Cpn10 treatment. The mucus in was predominantly blue in colour (e.g. FIG. 6). A systematic analysis of all bronchial glands available to be scored in i.v. treated sheep indicated that on average the bronchial gland lumen was 4.8% occluded with mucous (n=6 sheep).

### Discussion

Data from this trial clearly shows that systemically delivered Cpn10 has the capacity to ameliorate allergic inflammation which underlies the pathophysiology of asthma. Neutrophils, eosinophils and IgE antibodies are all known to be important components of inflammation associated with asthma. This trial shows that Cpn10 has significant dampening effects on eosinophilia and neutrophilia in the BAL and the level of HDM-specific IgE in serum after HDM challenge.

The assessment of Cpn10 administration to sheep sensitized to an asthma-inducing allergen relevant to human asthma has demonstrated a dose-responsive reduction in eosinophils accumulating in the bronchoalveolar space at 48 hr post-antigen challenge. Eosinophilia in the BAL of HDM-challenged sheep is similar to the level of eosinophils found in human asthmatics (Metzger et al, 1987). Another feature of the model used in this study is that sheep that display high specific IgE serum titres (i.e. allergic sheep) correlate with those individuals that generate elevated and prolonged BAL eosinophilia when challenged locally with HDM, compared with non-allergic and control sheep (Bischof, 2003).

The i.v. administration of Cpn10 has clear dose-dependent effects of attenuating allergic inflammation. The high (16 mg) Cpn10 dose was the most effective in reducing the percentage of neutrophils and eosinophils in BAL. The 16 mg Cpn10 dose also had a significant dampening effect on the level of blood eosinophils 24 and 48 h after challenge compared to eosinophils counted at same time-points for the 4 mg dose and the second HDM alone challenge trials.

Cpn10 appeared to have no untoward effects on sheep throughout the trial as their main clinical signs remained within the normal physiological range throughout the period of experiments. This indicates that Cpn10 is well tolerated in sheep at all doses used, irrespective of whether delivery is through i.v. or i.l. routes.

The histological analysis of the animal at post mortem resulted in some interesting observations. The i.v. administration of 4 mg of Cpn10 resulted in a marked reduction of a number of pathology parameters associated with allergic inflammation. In general i.v.-treated sheep had less infiltration of inflammatory cells into the airway walls, had fewer and less mature goblet cells, and had lower mucous content in the bronchial glandular lumens. There appeared to be little difference between the extent of pathology between vehicle (control) and Cpn10 infused lungs. In general intra-lung challenged sheep had pathology similar to that seen in HDM-challenged animals alone.

In summary, this trial clearly demonstrates that systemically delivered Cpn10 has the capacity to attenuate the key underlying inflammatory components of asthma.

### Example 2

### Compositions for Treatment

In accordance with the best mode of performing the invention provided herein, specific preferred compositions are outlined below. The following are to be construed as merely illustrative examples of compositions and not as a limitation of the scope of the present invention in any way.

### Example 2(A)

### Composition for Parenteral Administration

A composition for parenteral injection could be prepared to contain 0.05 mg to 5 g of a suitable agent or compound as disclosed herein in 10 mls to 2 liters of 1% carboxymethylcellulose.

Similarly, a composition for intravenous infusion may comprise 250 ml of sterile Ringer's solution, and 0.05 mg to 5 g of a suitable agent or compound as disclosed herein.

### Example 2(B)

### Composition for Oral Administration

A composition of a suitable agent or compound in the form of a capsule may be prepared by filling a standard two-piece hard gelatin capsule with 500 mg of the agent or compound, in powdered form, 100 mg of lactose, 35 mg of talc and 10 mg of magnesium stearate.

### REFERENCES

Busse W W, Lemanske R F Jr. Asthma. N Engl J Med 2001; 344:350-62.
Roche N, Chinet T C, Huchon G J. Allergic and nonallergic interactions between house dust mite allergens and airway mucosa. Eur Respir J 1997; 10:719-26.
Bischof R J, Snibson K, Shaw R, Meeusen E N T. Induction of allergic inflammation in the lungs of sensitized sheep after local challenge with house dust mite. Clin Exp Allergy 2003; 33:367-75.
Metzger W J, Zavala D, Richerson H B et al. Local allergen challenge and bronchoalveolar lavabe of allergic asthmatic lungs. Description of the model and local airway inflammation. Am Rev Respir Dis 1987; 135:433-40.

1. A method for inhibiting a hypersensitivity reaction in a subject, wherein said method comprises administering an effective amount of a polypeptide comprising an amino acid sequence selected from the group comprising SEQ ID NO: 3, 4, 7, 9, 11, 13 15 and 17.

2. The method of claim 1, wherein said polypeptide is encoded by a polynucleotide sequence selected from the group comprising SEQ ID NO: 6, 8, 10, 12, 14, 16 and 18.

3. The method of claim 1 wherein the hypersensitivity reaction involves the activation of cells selected from the group comprising: basophils, eosinophils, mast cells, neutrophils and lymphocytes.

4. The method of claim 1, wherein the hypersensitivity reaction involves activation of Toll-like receptor (TLR) signalling.

5. The method of claim 1, wherein the hypersensitivity reaction involves high levels of eosinophils and immunoglobulin E.

6. The method of claim 1, wherein the hypersensitivity reaction is an inflammatory reaction.

7. The method of claim 1, wherein the hypersensitivity reaction is selected from the group comprising: food allergy, dermatitis, allergic conjunctivitis, rhinitis, eczema, anaphylaxis and respiratory diseases associated with airway inflammation.

8. The method of claim 7, wherein the respiratory disease is selected from the group comprising: asthma, allergic asthma, intrinsic asthma, occupational asthma, ARDS (acute respiratory distress syndrome) and COPD (chronic obstructive pulmonary disease).

9. A method for treating or preventing a hypersensitivity reaction associated disorder in a subject, the method comprising administering to the subject an effective amount of a polypeptide comprising an amino acid sequence selected from the group comprising SEQ ID NO: 3, 4, 7, 9, 11, 13 15 and 17, wherein the polypeptide modulates signalling from a Toll-like receptor.

10. The method of claim 9, wherein the Toll-like receptor is selected from the group comprising: TLR1, TLR2, TLR3, TLR4, TLR5, TLR6, TLR7, TLR8, TLR9, and TLR10.

11. The method of claim 9, wherein said polypeptide sequence is encoded by a polynucleotide sequence selected from the group comprising SEQ ID NO: 6, 8, 10, 12, 14, 16 or 18.

12. The method of claim 9, wherein the method further comprises the administration of at least one additional agent.

13. The method of claim 12, wherein the agent is an immunomodulator.

14. The method of claim 13, wherein the immunomodulator is a type I interferon.

15. The method of claim 14, wherein the interferon is IFNα or IFNβ.

16. A composition for treating or preventing a hypersensitivity reaction associated disorder in a subject, the composition comprising an effective amount of a polypeptide comprising an amino acid sequence selected from the group comprising SEQ ID NO: 3, 4, 7, 9, 11, 13 15 and 17, together with an immunosuppressant agent.

17. The composition of claim 16, wherein the immunosuppressant agent is selected from the group comprising of anti-inflammatory compounds and bronchodilatory compounds.

18. The composition of claim 16, wherein the immunosuppressant agent is selected from the group comprising: cyclosporine, tacrolimus, sirolimus, mycophenolate mofetil, methotrexate, chromoglycalates, theophylline, leukotriene antagonist, and antihistamine, or a combination thereof.

19. The composition of claim 16, wherein the immunosuppressant agent is a specific antibody directed against B or T lymphocytes.

20. The composition of claim 19, wherein the specific antibody is directed against B or T lymphocyte surface receptors that mediate B or T lymphocyte activation.

21. The composition of claim 16, wherein the composition further comprises a steroid.

22. A method for treating or preventing a hypersensitivity reaction associated disorder in a subject, the method comprising administering an effective amount of the composition of claim 1.

23. The composition of claim 16 wherein the polypeptide sequence is encoded by a polynucleotide sequence selected from the group comprising SEQ ID NO: 6, 8, 10, 12, 14, 16 or 18.

24. The method of claim 1, wherein the method further comprises the administration of at least one additional agent.

25. The method of claim 24, wherein the agent is an immunomodulator.

26. The method of claim 25, wherein the immunomodulator is a type I interferon.

27. The method of claim 26, wherein the interferon is IFNα or IFNβ.


SEQUENCE LISTING (18 sequences)

SEQ ID NO: 1 - protein, 101 aa, Homo sapiens [sequence omitted]

SEQ ID NO: 2 - protein, 104 aa, Artificial Sequence
  modified human chaperonin 10 (Cpn10) with additional GSM tripeptide at N-terminus
GSMAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 3 - protein, 102 aa, Artificial Sequence
  modified human chaperonin 10 (Cpn10) with additional alanine (A) residue at N-terminus
AAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 4 - protein, 102 aa, Artificial Sequence
  modified human chaperonin 10 (Cpn10) with additional glycine (G) residue at N-terminus
GAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 5 - DNA, 309 bp, Homo sapiens
  wild-type human chaperonin 10 (Cpn10), heat shock protein 10 (Hsp10, HU-HSP10)
atggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 6 - DNA, 312 bp, Artificial Sequence
  modified human chaperonin 10 (Cpn10) with additional glycine (G) residue at N-terminus
atgggtgcgggccaggcgtttcgtaaatttctgccgctgtttgatcgtgtgctggttgaacgtagcgcggcggaaaccgtgaccaaaggcggcattatgctgccggaaaaaagccagggcaaagtgctgcaggcgaccgtggttgcggttggcagcggcagcaaaggcaaaggcggcgaaattcagccggtgagcgtgaaagtgggcgataaagtgctgctgccggaatatggcggcaccaaagtggtgctggatgataaagattattttctgttccgcgatggcgatattctgggcaaatatgtggattga

SEQ ID NO: 7 - protein, 86 aa, Artificial Sequence
  modified human chaperonin 10 (Cpn10) with deletion of mobile loop
AAGQAFRKFLPLFDRVLVERSAGKVLQATVVAVGSGSKGKGGEIQPVSVKVGDKVLLPEYGGTKVVLDDKDYFLFRDGDILGKYVD

SEQ ID NO: 8 - DNA, 264 bp, Artificial Sequence
  modified human chaperonin 10 (Cpn10) with deletion of mobile loop
atggcggcgggtcaggcgtttcgtaaatttctgccgctgtttgatcgtgttctggttgaacgtagcgcgggcaaagttctgcaggcgaccgttgttgcggttggtagcggcagcaaaggtaaaggcggtgaaattcagccggttagcgtgaaagtgggcgataaagttctgctgccggaatatggcggcaccaaagttgtgctggatgataaagattacttcctgttccgcgatggtgatatcctgggcaaatacgtggattaa

SEQ ID NO: 9 - protein, 95 aa, Homo sapiens
  modified human chaperonin 10 (Cpn10) with deletion of beta hairpin roof loop
AAGQAFRKFLPLFDRVLVERSAAETVTKGGIMLPEKSQGKVLQATVVAVGSGSQPVSVKVGDKVLLPEYGGTKVVLDDKDYFLFRDGDILGKYVD

SEQ ID NO: 10 - DNA, 291 bp, Artificial Sequence
  modified human chaperonin 10 (Cpn10) with deletion of beta hairpin roof loop
atggcggcgggtcaggcgtttcgtaaatttctgccgctgtttgatcgtgttctggttgaacgtagcgcggcggaaaccgttaccaaaggcggtattatgctgccggaaaaaagccagggtaaagttctgcaggcgaccgttgttgcggttggtagcggtagccagccggttagcgtgaaagtgggcgataaagttctgctgccggaatatggcggcaccaaagttgtgctggatgataaagattacttcctgttccgcgatggtgatatcctgggcaaatacgtggattaa

SEQ ID NO: 11 - protein, 79 aa, Artificial Sequence
  modified human chaperonin 10 (Cpn10) with deletion of both mobile loop and beta hairpin roof loop
AAGQAFRKFLPLFDRVLVERSAGKVLQATVVAVGSGSQPVSVKVGDKVLLPEYGGTKVVLDDKDYFLFRDGDILGKYVD

SEQ ID NO: 12 - DNA, 243 bp, Artificial Sequence
  modified human chaperonin 10 (Cpn10) with deletion of both mobile loop and beta hairpin roof loop
atggcagcaggacaagcgtttagaaagtttcttccactctttgaccgagtattggttgaaaggagtgctggaaaagtattgcaagcaacagtagtcgctgttggatcgggttctcaaccagttagcgtgaaagttggagataaagttcttctcccagaatatggaggcaccaaagtagttctagatgacaaggattatttcctatttagagatggtgacattcttggaaagtacgtagactga

SEQ ID NO: 13 - protein, 102 aa, Artificial Sequence
  modified human chaperonin 10 (Cpn10) with tripeptide deletion within mobile loop
AAGQAFRKFLPLFDRVLVERSAAETVTKGGIFIPEKSQGKVLQATVVAVGSGSKGKGGEIQPVSVKVGDKVLLPEYGGTKVVLDDKDYFLFRDGDILGKYVD

SEQ ID NO: 14 - DNA, 312 bp, Artificial Sequence
  modified human chaperonin 10 (Cpn10) with tripeptide deletion within mobile loop
atggcagcaggacaagcgtttagaaagtttcttccactctttgaccgagtattggttgaaaggagtgctgctgaaactgtaaccaaaggaggcattttcattccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 15 - protein, 102 aa, Artificial Sequence
  modified human chaperonin 10 (Cpn10) with tripeptide deletion within mobile loop
AAGQAFRKFLPLFDRVLVERSAAETVTKGGIIIPEKSQGKVLQATVVAVGSGSKGKGGEIQPVSVKVGDKVLLPEYGGTKVVLDDKDYFLFRDGDILGKYVD

SEQ ID NO: 16 - DNA, 312 bp, Artificial Sequence
  modified human chaperonin 10 (Cpn10) with tripeptide deletion within mobile loop
atggcagcaggacaagcgtttagaaagtttcttccactctttgaccgagtattggttgaaaggagtgctgctgaaactgtaaccaaaggaggcattataattccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 17 - protein, 110 aa, Artificial Sequence
  modified human chaperonin 10 (Cpn10) with tripeptide deletion within mobile loop
AAGQAFRKFLPLFDRVLVERSAAETVTKGGEEEPEKSQGKVLQATVVAVGSGSKGKGGEIQPVSVKVGDKVLLPEYGGTKVVLDDKDYFLFRDGDILGKYVDLEHHHHHH

SEQ ID NO: 18 - DNA, 336 bp, Artificial Sequence
  modified human chaperonin 10 (Cpn10) with tripeptide deletion within mobile loop
atggcagcaggacaagcgtttagaaagtttcttccactctttgaccgagtattggttgaaaggagtgctgctgaaactgtaaccaaaggaggcgaagaggaaccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagacctcgagcaccaccaccaccaccactga